(19) United States
(12) Patent Application Publication
(10) Pub. No.: US 2022/0023361 A1
(43) Pub. Date: Jan. 27, 2022

(54) COMPOSITION AGAINST RESISTANT ORGANISMS (MRSA)

(71) Applicant: FermentationExperts A/S, Bække (DK)

(72) Inventor: Søren Kjærulff, Copenhagen (DK)

(21) Appl. No.: 17/277,592

(22) PCT Filed: Sep. 18, 2019
(86) PCT No.: PCT/EP2019/075073

§ 371 Date: Mar. 18, 2021

(30) Foreign Application Priority Data
Sep. 19, 2018 (DK) PA 2018 00611

(51) Int. Cl. A61K 36/03; A61K 36/04; A61K 35/744; A61P 31/04

(52) U.S. Cl. CPC A61K 36/03; A61P 31/04; A61K 35/744; A61K 36/04

(57) ABSTRACT

A composition comprises: (i) at least one fermented seaweed material; (ii) at least one fermented plant material: or (iii) a combination of at least one fermented seaweed material and at least one fermented plant material; and at least one lactic acid bacterial strain, for treating, alleviating, suppressing and/or preventing growth of a resistant organism.

### TECHNICAL FIELD OF THE INVENTION

The present invention relates to a composition against resistant organisms. In particular, the present invention relates to a composition suitable for inhibiting, reducing, treating, alleviating, suppressing and/or preventing growth of a resistant organism.

### BACKGROUND OF THE INVENTION

Staphylococcus is a genus of Gram-positive bacteria in the family Staphylococcaceae in the order Bacillales. Under the microscope, they appear spherical (cocci), and form in grape-like clusters. Staphylococcus species are facultative anaerobic organisms (capable of growth both aerobically and anaerobically).

Staphylococcus includes at least 40 species. Of these, nine have two subspecies, one has three subspecies, and one has four subspecies. Most are harmless and reside normally on the skin and mucous membranes of humans and other organisms. Staphylococcus has been found to be a nectar-inhabiting microbe. Found worldwide, they are a small component of soil microbial flora

Staphylococcus is a natural part of the bacterial flora in about 50% of all people, and they are mainly found in the nose, in the respiratory tract, and on the skin.

Staphylococcus aureus is a member of the normal flora of the human body, frequently found in the nose, respiratory tract, and on the skin. It is often positive for catalase and nitrate reduction and is a facultative anaerobe. Although Staphylococcus aureus is not always pathogenic (and can commonly be found existing as a commensal), it is a common cause of skin infections including abscesses, respiratory infections such as sinusitis, and food poisoning. Pathogenic strains of Staphylococcus aureus often promote infections by producing virulence factors such as potent protein toxins, and the expression of a cell-surface protein that binds and inactivates antibodies.

Staphylococcus aureus infections can spread through contact with pus from an infected wound, skin-to-skin contact with an infected person, and contact with objects used by an infected person such as towels, sheets, clothing, or athletic equipment. Joint replacements put a person at particular risk of septic arthritis, staphylococcal endocarditis (infection of the heart valves), and pneumonia.

Diabetics, injection drug users, and individuals with heart conditions should take extra precautions to avoid coming into contact with Staphylococcus aureus, as they are at the highest risk. Preventive measures include washing hands often with soap and making sure to bathe or shower daily.

Staphylococcus aureus is a significant cause of chronic biofilm infections on medical implants, and the repressor of toxins is part of the infection pathway.

Staphylococcus aureus can lay dormant in the body for years undetected. Once symptoms begin to show, the host is contagious for another two weeks, and the overall illness lasts a few weeks. If untreated, though, the disease can be deadly. Deeply penetrating Staphylococcus aureus infections can be severe.

Methicillin-resistant Staphylococcus aureus (MRSA) refers to a group of Staphylococcus aureus that are genetically distinct from other strains of Staphylococcus aureus. MRSA is any strain of Staphylococcus aureus that has developed, through horizontal gene transfer and natural selection, multiple drug resistance to e.g. beta-lactam antibiotics. β-lactam antibiotics are a broad spectrum group which includes some penams—penicillin derivatives such as methicillin and oxacillin, and cephems such as the cephalosporins.

This emergence of this multiple drug resistant and antibiotic-resistant strains of Staphylococcus aureus, methicillin-resistant Staphylococcus aureus (MRSA) is a worldwide problem in clinical medicine and is responsible for several difficult-to-treat infections, in particular in humans. Despite much research and development, no vaccine for Staphylococcus aureus has been approved and treatment is difficult.

MRSA began as a hospital-acquired infection but has become community-acquired as well as livestock-acquired. The terms HA-MRSA (healthcare-associated or hospital-acquired MRSA), CA-MRSA (community-associated MRSA) and LA-MRSA (livestock-associated) reflect this.

Thus, as mentioned above, in case of infection with a resistant organism, such as MRSA, it may be difficult, if possible, to treat such infection, and the treatment may be expensive, involve various side effects, insufficient and reoccurring treatment may be required, and there is a risk of inducing further resistance to medical treatments.

Hence, an improved composition for inhibiting, reducing, treating, alleviating, suppressing and/or preventing growth of a resistant organism would be advantageous, and in particular a more efficient and/or reliable composition, which is inexpensive, reduce or even avoid various side effects, reduce or even avoid repeated reoccurring treatments, and reduce or avoid the risk of inducing additional resistance to medical treatments, would be advantageous.

### SUMMARY OF THE INVENTION

Thus, an object of the present invention relates to an improved composition for inhibiting, reducing, treating, alleviating, suppressing and/or preventing growth of a resistant organism.

In particular, it is an object of the present invention to provide a composition that solves the above-mentioned problems of the prior art with lack of efficiency and/or reliability, which composition, is inexpensive, reduce or even avoid various side effects, reduce or even avoid repeated reoccurring treatments, and/or reduce or avoid the risk of additional resistance to medical treatments.

Thus, one aspect of the invention relates to a composition comprising:
(i) at least one fermented seaweed material;
(ii) at least one fermented plant material: or
(iii) a combination of at least one fermented seaweed material and at least one fermented plant material;
   and at least one lactic acid bacterial strain, for use in treating, alleviating, suppressing and/or preventing growth of a resistant organism.

A further aspect of the present invention relates to an animal feed product comprising a composition according to the present invention.

Yet, another aspect of the present invention relates to the use of the composition according to the present invention for treating, alleviating, suppressing and/or preventing growth of a resistant organism.

### DETAILED DESCRIPTION OF THE INVENTION

Treatment of infections caused by resistant organisms, such as MRSA (methicillin-resistant Staphylococcus aureus), which is a form of contagious bacterial infection that is resistant to numerous antibiotics including methicillin, amoxicillin, penicillin, and oxacillin. This resistance makes it challenging to treat. Often referred to as a superbug, MRSA infection may begin as a skin sore, pimple, or boil, before becoming serious, potentially harmful, and sometimes fatal.

Even this multi-resistance, the inventors of the present invention surprising found a composition that showed positive activity in treating, alleviating, suppressing and/or preventing growth of a resistant organism a resistant organism, like MRSA.

Thus, a preferred embodiment of the present invention relates to a composition comprising:
(i) at least one fermented seaweed material;
(ii) at least one fermented plant material: or
(iii) a combination of at least one fermented seaweed material and at least one fermented plant material;
   and at least one lactic acid bacterial strain, for use in treating, alleviating, suppressing and/or preventing growth of a resistant organism.

In another preferred embodiment of the present invention wherein the composition is consisting essentially of:
(i) at least one fermented seaweed material;
(ii) at least one fermented plant material: or
(iii) a combination of at least one fermented seaweed material and at least one fermented plant material;
   and at least one lactic acid bacterial strain, for use in treating, alleviating, suppressing and/or preventing growth of a resistant organism.

In the context of the present invention, the term “comprising”, which may be synonymous with the terms “including”, “containing” or “characterized by”, relates to an inclusive or open-ended listing of features and does not exclude additional, unrecited features or method steps. The term “comprising” leaves the claim open for the inclusion of unspecified ingredients even in major amounts.

In the context of the present invention, the term “consisting essentially of”, relates to a limitation of the scope of a claim to the specified features or steps and those features or steps, not mentioned and that do not materially affect the basic and novel characteristic(s) of the claimed invention.

In an embodiment of the present invention, the composition according to the present invention may be used as an ingredient in a feed product or in a food product, resulting in a control of the growth of resistant organisms, e.g. inhibiting, reducing and/or suppressing the growth or even killing the resistant organism present in the feed product or in the food product.

In another embodiment of the present invention, the composition may be used for human or animal administration. When the composition is used for human or animal administration it may be used as a product as such, or it may be used as an ingredient added to the feed product or food product.

The at least one seaweed material forming part of the composition may preferably comprise at least one fermented seaweed material. Furthermore the at least one seaweed material may preferably be a fermented seaweed material.

The term “at least one seaweed material” implies that different seaweed materials may be used. In an embodiment of the present invention the composition comprises at least one seaweed material, such as at least two seaweed materials, e.g. at least three seaweed materials, such as at least four seaweed materials.

When the composition comprises two or more seaweed materials, the seaweed materials may be of different origin and/or the seaweed materials may be of same origin but one being fermented and another non-fermented. Preferably, the main part (w/w) of the seaweed material in the composition may be fermented seaweed material.

In an embodiment of the present invention wherein the seaweed material may be a unicellular alga or a multicellular macroalgae.

In a further embodiment of the present invention the multicellular macroalgae may be selected from brown macroalgae, red macroalgae, and/or green macroalgae. Preferably the microalgae may be brown macroalgae and/or red macroalgae. Even more preferably, the microalgae may be brown macroalgae.

In another embodiment of the present invention the brown macroalgae may be selected from one or more of kelps, Saccharina latissimi (Laminaria saccharina), Laminaria digitate, Ascophyllum nodosum, Laminaria hyperborean, or a mixture hereof.

The at least one plant material forming part of the composition may preferably comprise at least one fermented plant material. Furthermore, the at least one plant material may be a fermented plant material.

In the present context, the term “plant material” relates to capable of performing photosynthesis.

The term “at least one plant material” implies that different plant materials may be used.

In an embodiment of the present invention the composition comprises at least one plant material, such as at least two plant materials, e.g. at least three plant materials, such as at least four plant materials.

When the composition comprises two or more plant materials, the plant materials may be of different origin and/or the plant materials may be of same origin but one being fermented and another being non-fermented. Preferably, the main part (w/w) of the plant material in the composition may be fermented plant material.

In an embodiment of the present invention the plant material may be selected from at least one proteinaceous plant material. The proteinaceous plant material may be selected from eudicot plants, angiosperm plants, and/or rosid plants.

Preferably the proteinaceous plant material may be selected from Brassicale plants.

In an embodiment of the present invention the Brassicale plants is selected from the Brassicaceae family or the Cruciferae family.

In a further embodiment of the present invention the Brassicaceae family or the Cruciferae family may be selected from at least one of a Brassica genus; sun flower; palm; soya, field beans, Lupins; or a combination hereof. Preferably, at least one Brassica genus may be selected from one or more species such as Brassica napus; Brassica oleracea; Brassica campestris; Brassica nigra; Sinapis alba (Brassica alba); Brassica juncea; Brassica rapa or mixtures hereof.

In yet an embodiment of the present invention the at least one Brassica genus may be selected from the group consisting of: including rape, rapeseed, canola, cabbage, broccoli, cauliflower, kale, Brussels sprouts, collard greens, savoy, kohlrabi, gai lan, white mustard, Indian mustard, Chinese mustard, and black mustard seed powder.

The composition may comprise a combination of at least one seaweed material as defined above, and at least one plant material as defined above.

Preferably, the combination of at least one seaweed material as defined above, and at least one plant material as defined above may be a combination of at least one fermented seaweed material and at least one fermented plant material.

The fermentation process for providing the at least one fermented seaweed and/or the at least one fermented plant material may preferably be a solid stage fermentation.

The solid stage fermentation may be performed at a humidity in the range of 30-50% (w/w) humidity, such as in the range of 35-45% (w/w) humidity, e.g. in the range of 38-42% (w/w) humidity, such as about 40% (w/w) humidity.

In the present context the term “fermented material” (a plant material, a seaweed material or a combination of the plant material and the seaweed material) relates to a controlled metabolic process of the material by adding a predetermined amount of fermenting microorganisms to the material allowing the microorganism and the material to interact breaking down the material.

The fermenting microorganisms may be a health-enhancing microorganism.

Hence, in an embodiment of the present invention the composition may comprise one or more health-enhancing microorganism, preferably the health-enhancing microorganism may be a health-enhancing yeast and/or a health-enhancing bacterium, even more preferably the health-enhancing microorganism may be a health-enhancing bacterium.

The health-enhancing bacteria may comprise one or more probiotics. The one or more probiotics and/or the one or more health-enhancing microorganism may comprise at least one lactic acid bacterial strain.

In the context of the present invention, the term “probiotic” relates to live microorganisms that when administered in adequate amounts, confer a health benefit on the host.

In a preferred embodiment of the present invention the at least one lactic acid bacterial strain may be selected from the group consisting of the genus Enterococcus, Lactobacillus, Pediococcus, Lactococcus, or Bifidobacterium or combinations thereof.

In a further embodiment of the present invention the one or more lactic acid bacterial stain(s) may be selected from the group consisting of Pediococcus pentosaceus; Pendiococcus acidilactici; Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentoses, Lactobacillus vaginalis, Lactobacillus xylosus and a combination thereof.

In an embodiment of the present invention the health-enhancing microorganism may be the main microorganism present in the composition. Preferably, the main microorganism is a lactic acid bacterium. Even more preferably the main microorganism may be selected from the groups consisting of Pediococcus pentosaceus; Pendiococcus acidilactici, Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentoses, Lactobacillus vaginalis, and Lactobacillus xylosus; preferably, the main lactic acid bacteria present in the composition is Lactobacillus plantarum.

In the context of the present invention the term “main microorganism” relates to the microorganism present in highest amount, determined on a weight/weight ratio.

During fermentation a group of microorganisms may be used for fermenting the plant material to provide a co-fermentation. The co-fermentation may be a mixture of different microorganisms (such as a mixture of yeasts, fungus, and/or bacteria) or a mixture of different bacteria. Preferably the co-fermentation comprises a mixture of different bacterial strains.

In an embodiment of the present invention the composition comprises one or more bacterial strains, e.g. two or more bacterial strains, such as three or more bacterial strains, e.g. four or more bacterial strains, such as 7 or more bacterial strains, e.g. 10 or more bacterial strains, such as 15 or more bacterial strains, e.g. 20 or more bacterial strains, such as 25 or more bacterial strains, e.g. 30 or more bacterial strains, such as 35 or more bacterial strains, e.g. 40 or more bacterial strains. Preferably, the bacterial strains may be one or more lactic acid bacterial strains.

In yet an embodiment of the present invention the composition comprises one or more lactic acid bacterial strains, e.g. two or more lactic acid bacterial strains, such as three or more lactic acid bacterial strains, e.g. four or more lactic acid bacterial strains, such as 7 or more lactic acid bacterial strains, e.g. 10 or more lactic acid bacterial strains, such as 15 or more lactic acid bacterial strains, e.g. 20 or more lactic acid bacterial strains, such as 25 or more lactic acid bacterial strains, e.g. 30 or more lactic acid bacterial strains, such as 35 or more lactic acid bacterial strains, e.g. 40 or more lactic acid bacterial strains.

In yet an embodiment of the present invention the composition consist essentially of one or more lactic acid bacterial strains, e.g. two or more lactic acid bacterial strains, such as three or more lactic acid bacterial strains, e.g. four or more lactic acid bacterial strains, such as 7 or more lactic acid bacterial strains, e.g. 10 or more lactic acid bacterial strains, such as 15 or more lactic acid bacterial strains, e.g. 20 or more lactic acid bacterial strains, such as 25 or more lactic acid bacterial strains, e.g. 30 or more lactic acid bacterial strains, such as 35 or more lactic acid bacterial strains, e.g. 40 or more lactic acid bacterial strains.

In a further embodiment of the present invention the one or more lactic acid bacteria stain(s) may be selected from the group consisting of one or more of Enterococcus faecium MCIMB 30122, Lactobacillus rhamnosus NCIMB 30121, Pediococcus pentosaceus HTS (LMG P-22549), Pendiococcus acidilactici NCIMB 30086 and/or Lactobacillus plantarum LSI (NCIMB 30083).

In order to provide the desired effects, the composition should have a high content of viable lactic acid bacteria. In an embodiment of the present invention the composition comprises one or more lactic acid bacterial strain(s) in a total amount in the range of 10⁵-10¹² CFU per gram of the composition, such as in the range of 10⁶-10¹² CFU per gram, e.g. in the range of 10²-10¹¹ CFU per gram, such as in the range of 10⁸-10¹¹ CFU per gram, e.g. in the range of 10⁹-10¹⁰ CFU per gram.

During fermentation, various metabolites may be formed, which in combination with the fermenting organism, and the at least one plant material, and/or the at least one seaweed material, may assist for use in the inhibiting, reducing and/or suppressing growth and/or the treatment, prophylaxis and alleviation of an infection with a resistant organism, such as a multiple resistant organism, such as MRSA.

In an embodiment of the present invention, the composition may be used for the treatment, alleviation, suppression, and/or prevention of growth of a resistant organism in a human or in an animal or in a room.

In a further embodiment of the present invention the room may be a hospital room, a healthcare setting, a barn, a stable, a prison, a nursing home or a cage.

As mentioned above, it was surprising to the inventors that the composition according to the present invention was active in controlling growth of a resistant organism, such as a multiple resistant organism, such as MRSA.

In an embodiment of the present invention the resistant organism may be a resistant bacterium.

In a further embodiment of the present invention the resistant bacterium may be a resistant Staphylococcus aureus.

Preferably, the resistant Staphylococcus aureus is a methicillin-resistant Staphylococcus aureus (MRSA) or an oxacillin-resistant Staphylococcus aureus (ORSA).

Originally, MRSA (methicillin-resistant Staphylococcus aureus) began as a hospital-acquired infection but has become community-acquired as well as livestock-acquired. The resistant organism according to the present invention may be a HA-MRSA (healthcare-associated or hospital-acquired MRSA), a CA-MRSA (community-associated MRSA) and/or a LA-MRSA (livestock-associated MRSA).

In the context of the present invention the term “healthcare-associated MRSA” or “hospital-acquired MRSA” relates to MRSA appearing in hospitals and other healthcare settings, representing a growing public health problem in the world. Many patients in hospitals are carriers of HA-MRSA but do not have disease symptoms. While these patients are the most common source of the bacteria, transmission occurs when healthcare workers' hands touch other patients who are HA-MRSA carriers. Failure to wash contaminated hands can foster spread of the bacteria. It's important to have good hygiene practices in the hospital—for everyone—to prevent these infections from spreading. Other sources of transmission in healthcare settings include open wounds, catheters, or breathing tubes.

In the context of the present invention the term “community-associated MRSA” relates to infections in healthy people living in the community, who have not been in hospital or had any medical procedures. community-associated MRSA can spread to others who are in close contact with a person who has community-associated MRSA, especially those who share the same household. community-associated MRSA usually causes skin infections, for example boils, that often occur again following initial treatment. Less commonly, it can cause serious infections like pneumonia and septicaemia (blood poisoning).

In the context of the present invention the term “livestock-associated MRSA” relates to infections in an animal. livestock-associated Methicillin-resistant Staphylococcus aureus can cause serious bloodstream infections in people and has been shown to cause skin and soft-tissue infections in humans. In Denmark, pigs are believed to be the primary source of LA-MRSA where it has been found on 60 percent of farms. LA-MRSA can spread from the farm animals to humans (and other animals) through direct contact with the animals, through contaminated meat that's produced from the animals, and possibly through air and water near industrial hog operations.

The composition according to the present invention the composition may comprise a lactic acid concentration of at least 50 mM, such as at least 100 mM, such as 100-1000 mM, such as 100-500 mM, such as 100-300 mM, such as 100-200 mM, such as 150-500 mM, such as 200-500 mM or such as 300-500, mM lactic acid.

In an embodiment of the present invention the composition may comprise a pH-value below pH 6.5, such as below pH 6.0, e.g. below pH 5.5, such as below pH 5.0, e.g. in the range of pH 3.0-6.5, such as in the range of pH 3.0-6.0, e.g. in the range of pH 3.1-5.5, such as in the range of pH 5.0-3.2, such as in the range 3.3- 4-2, such as 3.4-4.0, such as 3.5-3.8, such as 3.7-4.2, such as 3.7-4.0, or such as 3.8-4.2.

The composition according to the present invention may preferably comprises a fibrous material. Preferably the composition comprises the fibrous material originating from the plant material and/or the seaweed material.

In an embodiment of the present invention the fibrous material may originate from the at least one seaweed material, the at least one plant material or the combination of at least one seaweed material and at least one plant material.

In an embodiment of the present invention the composition comprises more than 5 g fibrous material per kg dried composition, such as more than 10 g fibrous material per kg dried composition, e.g. more than 15 g fibrous material per kg dried composition, such as more than 20 g fibrous material per kg dried composition, e.g. more than 25 g fibrous material per kg dried composition, such as more than 50 g fibrous material per kg dried composition, e.g. more than 75 g fibrous material per kg dried composition, such as more than 100 g fibrous material per kg dried composition, e.g. more than 150 g fibrous material per kg dried composition, such as more than 200 g fibrous material per kg dried composition, e.g. more than 250 g fibrous material per kg dried composition, such as more than 300 g fibrous material per kg dried composition.

The composition according to the present invention may preferably comprise a starch material. Preferably the composition comprises the starch material originating from the plant material and/or the seaweed material.

In an embodiment of the present invention the starch material may originate from the at least one seaweed material, the at least one plant material or the combination of at least one seaweed material and at least one plant material.

The composition (preferably the fermented composition) may be a dried composition comprises a range of 30-70% (w/w); such as a range of 40-60% (w/w); e.g. about 50% (w/w) of the dry composition has a particle size below 0.5 mm and a range of 30-70% (w/w); such as a range of 40-60% (w/w); e.g. about 50% (w/w) of the dry composition has a particle size above 0.5 mm.

Preferably, the composition may be a fermented composition comprising at least one fermented seaweed material, at least one fermented plant material, or the combination of at least one fermented seaweed material and at least one fermented plant material.

In an embodiment of the present invention wherein the composition may comprise more than 5 g starch material per kg dried composition, such as more than 10 g starch material per kg dried composition, e.g. more than 15 g starch material per kg dried composition, such as more than 20 g starch material per kg dried composition, e.g. more than 25 g starch material per kg dried composition, such as more than 50 g starch material per kg dried composition, e.g. more than 75 g starch material per kg dried composition, such as more than 100 g starch material per kg dried composition, e.g. more than 150 g starch material per kg dried composition, such as more than 200 g starch material per kg dried composition, e.g. more than 250 g starch material per kg dried composition, such as more than 300 g starch material per kg dried composition.

In an embodiment of the present invention the at least one fermented seaweed material, at least one fermented plant material, or the combination of at least one fermented seaweed material is not subjected sterilization in order to eliminate, remove, kill, or deactivate all forms of life in the the at least one fermented seaweed material, at least one fermented plant material, or the combination of at least one fermented seaweed material before the fermentation.

In a further embodiment of the present invention, the inoculum may be provided with a concentration of lactic acid bacteria in the inoculum sufficient to outgrowth any bacteria, yeast or moulds present in the product of step (b).

In an embodiment of the present invention the fermentation process may be performed at a temperature in the range of 15-40° C., such as 25-35° C., such as 30-40° C., such as 15-20° C. or such as 40-45° C.

In a further embodiment of the present invention the fermentation of the at least one plant material may be performed for a period of at least 3 days, e.g. for at least 5 days, such as 5-12 days, such as 5-10 days, such as 5-7 days or such as 8-10 days.

In yet an embodiment of the present invention the fermentation of the at least one seaweed material may be performed for a period in the range 2-40 days, such as for a period in the range of 4-30 days, e.g. for a period in the range of 8-20 days, such as for a period in the range 10-12 days, or for at least 2 days, such as for at least 3 days, e.g. for at least 5 days, such as for at least 8 days, e.g. for at least 10 days, such as for at least 12 days, e.g. for at least 15 days, such as for at least 18 days.

Preferably, the fermentation of the at least one plant material and the fermentation of the at least one seaweed material may be performed jointly. Further details on the fermentation procedure may be found in WO 2014/206419, which is hereby incorporated by reference.

If fermentation is performed jointly with the plant material and the seaweed material, the fermentation of the seaweed material may be started first and the plant material may be added subsequently. In an embodiment of the present invention the seaweed material may be subjected to fermentation for 1-15 days; such as 3-12; e.g. 5-10; such as about 7 days before the plant material is added. When the plant material is added to the fermented seaweed material the fermentation may be continued with both the seaweed and the plant material for 2-20 days; such as for 4-16 days; e.g. for 8-13 days; e.g. for about 11 days.

During fermentation the pH of the material fermented may preferably drop within the first 24 hours of fermentation to pH 6 or below, such as pH 5 or below, e.g. pH 4.5 or below, such as pH 4.2 or below. Preferably, the drop in pH may be provided by the fermentation process, preferably no addition of acid may be made.

It may be preferred that the composition according to the present invention may be a dried composition. Preferably the composition according to the present invention has a moisture content in the range of 4%-15%% (w/w), such as in the range of 5%-14% (w/w), e.g. in the range of 6%-13% (w/w), such as in the range of 7%-12% (w/w), e.g. in the range of 8%-11% (w/w), such as in the range of 9%-10% (w/w).

The drying of the fermented ingredient may preferably be a gentle drying method in order to maintain the active potential of the fermented ingredient. An example of a gentle drying method may be as described in WO 2013/029632.

In an embodiment of the present invention the dried fermented composition comprises a range of 30-70% (w/w); such as a range of 40-60% (w/w); e.g. about 50% (w/w) of the dry fermented composition has a particle size below 0.5 mm and a range of 30-70% (w/w); such as a range of 40-60% (w/w); e.g. about 50% (w/w) of the dry fermented composition has a particle size above 0.5 mm.

In a further embodiment of the present invention the dried fermented composition comprises at least 2, preferably at least 3, even more preferably at least 4 of the following criteria:
a) 1-10% (w/w), such as about 5% (w/w), of the dry fermented composition has a particle size above 1.0 mm;
b) 45-55% (w/w), such as about 50% (w/w), of the dry fermented composition has a particle size between 0.5-1.0 mm;
c) 30-40% (w/w), such as about 50% (w/w), of the dry fermented composition has a particle size between 0.25-0.5 mm; and/or
d) 5-15% (w/w), such as about 10% (w/w), of the dry fermented composition has a particle size below 0.25 mm.

In the present context the term “about” relates to a variation on the stated amount of 10% or less, such as 5% or less, e.g. 1% or less.

The selection of the various particle sizes may be determined by sieving as known to the skilled person.

Before fermentation, the at least one plant material and/or the at least one seaweed material may have an average maximum diameter of 5 cm, such as an average maximum diameter of 4 cm such as an average maximum diameter of 3 cm, such as an average maximum diameter of 2 cm, such as an average maximum diameter of 1 cm, such as an average diameter in the range 25 pm to 5 cm, such as 0.1 mm to 5 cm, such as an average diameter in the range of 0.5 mm to 5 cm, such as an average diameter in the range 0.5 mm to 2 cm.

The composition according to the present invention may be used as an ingredient for an animal feed product.

Thus, an aspect of the present invention relates to an animal feed product comprising a composition according to the present invention, Preferably, for use in treating, alleviating, suppressing and/or preventing growth of a resistant organism

When using the composition according to the present invention as an ingredient for an animal feed product, the fermented composition may be mixed with the basic feed compound as a free-flowing powder or before animal feed pellets are formed, resulting in animal feed pellets comprising the fermented ingredient. Preferably the fermented ingredient is mixed with the basic feed compound as a dry fermented ingredient.

The composition according to the present invention may assist in the enhancing the nutritional and antibiotic benefits of the animal feed product.

In another embodiment of the present invention the composition may be formulated into a powder, a tablet, a pill, a sachet, a crème, an ointment, or an oil, or a suspension.

Preferably, the composition may be provided as powder, a tablet, a pill, a sachet, or a suspension, an extruded wet cake, or a mash for the oral administration. Preferably, the composition may be provided as a pill, a tablet, or a suspension for rectal administration.

Preferably, the composition is provided as a crème, an ointment, an oil, or a suspension for topical administration.

When, the composition may be used for the treatment, alleviation, suppression, and/or prevention of growth of a resistant organism in a room the composition may be provided as a crème, an ointment, an oil, or a suspension for application in a room.

The composition may be used preventively in a room to avoid the development of resistant organisms or the composition may be used in a room contaminated with resistant organism for treating, alleviating, suppressing and/or preventing growth of a resistant organism.

In an embodiment of the present invention the composition may be applied on the surfaces in a room. Preferably, the composition may be applied on the surfaces in a room by spraying, painting, or splashing.

A preferred embodiment of the present invention relates to the use of the composition according to the present invention for treating, alleviating, suppressing and/or preventing growth of a resistant organism.

Preferably, the composition may be for the treatment, alleviation, suppression, and/or prevention of growth of a resistant organism in a human or in an animal or in a room.

In an embodiment of the present invention the composition according to the present invention maybe suitable for treating, alleviating, suppressing and/or preventing growth resistant organism in a human or in an animal, such as pigs, broiler chickens, roaster chickens, lying hens, turkey; ducks, geese, quails, cows, dogs, sheep, goats, cats and horses.

In the context of the present invention, the term “treatment” relates to the use of the composition according to the present invention, in an attempt to cure or mitigate a disease, a condition, or an injury in a human or an animal.

The term “alleviation” used in the present invention relates to the action of the composition according to the present invention, to make a disease, a condition or an injury less intense and/or reduce symptoms in a human or an animal.

In the context of the present invention the term “prophylaxis” relates to the use of the composition according to the present invention, in an attempt to prevent a disease, a condition or an injury in a human or an animal, and/or for the protective treatment of a human or an animal.

In the present context the animal relates to pigs, broiler chickens, roaster chickens, lying hens, turkey; ducks, geese, quails, cows, dogs, sheep, goats, cats and horses.

The composition according to the present invention may be used directly or as part of or as an ingredient of another product.

A preferred embodiment of the present invention relates to a feed product comprising the composition according to the present invention.

In the event the composition according to the present invention may be used as part of a feed product or as an ingredient in a feed product, the feed product may comprise a content of the composition in the range of 0.5-25% (w/w), such as in the range of 1-20% (w/w), e.g. in the range of 2-18% (w/w), such as in the range of 3-16% (w/w), e.g. in the range of 5-15% (w/w), such as in the range of 7-12% (w/w), e.g. in the range of 8-10% (w/w).

In a preferred embodiment of the present invention the composition may provide inhibiting, reducing and/or suppressing effect on the growth of resistant organisms in a 10 food product and/or in a feed product as well as inhibiting, reducing and/or suppressing effect (or treating, alleviating and/or preventing growth) of at least one resistant organism in a human or animal consuming or using the composition.

When the composition comprises a fermented seaweed material; a fermented plant material, or the combination of a fermented seaweed material and a fermented plant material, the fermentation process may be performed by adding an inoculum comprising the fermenting microorganisms according to the present invention. Preferably the fermentation process may be an anaerobic fermentation process. Even more preferably the fermentation process for providing a composition comprising fermented plant material and/or fermented seaweed according to the present invention may be an anaerobic homolactic fermentation process.

The fermented seaweed material and/or the fermented plant material, may preferably not be subjected to sterilisation in order to maintain the native metabolic nature of the materials.

The inoculum may be provided with a concentration and/or in an amount of lactic acid bacteria in the inoculum sufficient to outgrow any bacteria, yeast or moulds present in the product seaweed material and/or the plant material.

Specific conditions for providing and fermenting the fermented seaweed material; the fermented plant material, or the combination of fermented seaweed material and fermented plant material, may be as described in WO 2008/006382; WO 2014/206419; or PCT/EP2016/076952. WO 2008/006382; WO 2014/206419; or PCT/EP2016/076952 are hereby incorporated by reference.

It should be noted that embodiments and features described in the context of one of the aspects of the present invention also apply to the other aspects of the invention.

All patent and non-patent references cited in the present application, are hereby incorporated by reference in their entirety.

### REFERENCES

WO 2008/006382

WO 2014/206419

PCT/EP2016/076952

1. A composition comprising:
(i) at least one fermented seaweed material;
(ii) at least one fermented plant material: or
(iii) a combination of at least one fermented seaweed material and at least one fermented plant material;
and at least one lactic acid bacterial strain, for use in treating, alleviating, suppressing and/or preventing growth of a resistant organism.

2. The composition according to claim 1, wherein the composition comprises one or more lactic acid bacterial strains, such as two or more lactic acid bacterial strains, such as three or more lactic acid bacterial strains, such as four or more lactic acid bacterial strains, such as 7 or more lactic acid bacterial strains, such as 10 or more lactic acid bacterial strains, such as 15 or more lactic acid bacterial strains, such as 20 or more lactic acid bacterial strains, such as 25 or more lactic acid bacterial strains, such as 30 or more lactic acid bacterial strains, such as 35 or more lactic acid bacterial strains, such as 40 or more lactic acid bacterial strains.

3. The composition according to claim 1, wherein the at least one seaweed material is performed for at least 2 days.

4. The composition according to claim 1, wherein the seaweed material is a unicellular alga or a multicellular macroalgae and wherein the multicellular macroalgae is selected from brown macroalgae and/or red macroalgae.

5. The composition according to claim 4, wherein the brown macroalgae is selected from one or more of kelps, Saccharina latissimi (Laminaria saccharina), Laminaria digitate, Ascophyllum nodosum, Laminaria hyperborean, or a mixture hereof.

6. The composition according to claim 1, wherein the resistant organism is a resistant bacterium.

7. The composition according to claim 6, wherein the resistant bacterium is a resistant Staphylococcus aureus.

8. The composition according to claim 7, wherein the resistant Staphylococcus aureus is a methicillin-resistant Staphylococcus aureus (MRSA) or a oxacillin-resistant Staphylococcus aureus (ORSA).

9. The composition according to claim 1, wherein the composition is for the treatment, alleviation, suppression, and/or prevention of growth of a resistant organism in a human or in an animal or in a room, wherein the room may be a hospital room, a barn, a stable, a prison, a nursing home or a cage.

10. The composition according to claim 1, wherein the composition comprises a fibrous material.

11. An animal feed product comprising a composition according to claim 1.

12. The animal feed product according to claim 11, wherein the animal feed product is mixed with a basic feed compound and formed as a free-flowing powder or as an animal feed pellet.
